(19) United States
(12) Patent Application Publication
(10) Pub. No.: US 2022/0257614 A1
(43) Pub. Date: Aug. 18, 2022

(54) USE OF 12-LIPOXYGENASE INHIBITORS IN THE TREATMENT OF COVID-19

(71) Applicant: New York Medical College, Valhalla, NY (US)

(72) Inventor: Jerry L. NADLER, Briarcliff Manor, NY (US)

(21) Appl. No.: 17/672,004

(22) Filed: Feb. 15, 2022

Related U.S. Application Data
(60) Provisional application No. 63/149,861, filed on Feb. 16, 2021.

(51) Int. Cl. A61K 31/635; A61K 31/7088; C07K 16/40; A61P 31/14

(52) U.S. Cl. CPC A61K 31/635; A61P 31/14; C07K 16/40; A61K 31/7088

(57) ABSTRACT

The present disclosure provides methods of treatment of coronavirus disease 2019 (COVID-19) based on the administration of 12-LO (12-LO) inhibitors.

### CROSS-REFERENCE TO RELATED APPLICATION

This application claims benefit and priority to U.S. Provisional Application No. 63/149,861, filed Feb. 16, 2021, which is incorporated herein by reference in its entirety.

### TECHNICAL FIELD

The present disclosure provides methods of treatment of coronavirus disease 2019 (COVID-19) based on the administration of 12-lipoxygenase (12-LO) inhibitors.

### BACKGROUND

Since the COVID-19 outbreak in December 2019, a worldwide pandemic has developed. So far, millions have been infected and hundreds of thousands of deaths have been reported. Accordingly, as the numbers continue to escalate, treatment methods for COVID-19 have become a top priority worldwide.

### SUMMARY

The presently disclosed subject matter provides a method of treating or preventing the symptoms associated with COVID-19. The method includes administering safely to a subject in need thereof a therapeutically effective amount of a arachidonic acid 12-lipoxygenase (12-LO) inhibitor. While the disclosure below is directed to treatment of COVID-19, which arises from infection with SARS-CoV-2, the disclosure applies equally as well to treatment or prevention of other coronavirus infections.

The presently disclosed subject matter provides a method for preventing, or alleviating, the symptoms associated with COVID-19 through administration of a 12-LO inhibitor in a therapeutically effective amount to a subject infected with or suspected of being infected with SARS-CoV-2. Such subjects may be identified as those having tested positive for infection with SARS-CoV-2 and/or those having COVID-19 symptoms. Such symptoms include, for example, fever, chills, cough, shortness of breath or difficulty breathing, fatigue, muscle or body aches, headache, new loss of taste or smell, sore throat, congestion or runny nose, nausea or vomiting and diarrhea.

Pharmaceutical compositions and formulations for use in treatment of COVID-19 include pharmaceutical compositions of a 12-LO inhibitor, alone or in combination with one or more additional therapeutic agents, in a mixture with a physiologically compatible carrier, which can be administered to a subject, for example, a human subject, for therapeutic treatment. The presently disclosed pharmaceutical compositions can be administered using a variety of methods known in the art depending on the subject and/or the severity of the COVID-19. In an aspect, the 12-LO inhibitor is administered, for example, orally, intranasally, by inhalation or intravenously.

The presently disclosed subject matter also includes the use of a 12-LO inhibitor, in the manufacture of a medicament for treatment of COVID-19. Regardless of the route of administration selected, the 12-LO inhibitor pharmaceutical compositions are formulated into pharmaceutically acceptable dosage forms such as described below or by other conventional methods known to those of skill in the art.

Actual dosage levels of the 12-LO inhibitor can be varied to obtain an amount of the active ingredient that is effective to achieve the desired therapeutic response for a particular subject without being toxic to the subject. The selected dosage level will depend on a variety of factors including the route of administration, the time of administration, the rate of excretion, the duration of the treatment, other drugs used in combination with the 12-LO inhibitor, the age, sex, weight, condition, general health, and prior medical history of the patient being treated, and like factors well known in the medical arts. A physician having ordinary skill in the art can readily determine and prescribe the effective amount of a given 12-LO inhibitor-containing pharmaceutical composition.

The presently disclosed compositions of a 12-LO inhibitor can be assembled into kits or pharmaceutical systems for use in treating COVID-19. In some embodiments, the presently disclosed kits or pharmaceutical systems include a 12-LO inhibitor in unit dosage form. In further embodiments, the 12-LO inhibitor can be present together with a pharmaceutically acceptable solvent, carrier, excipient, or the like, as described herein. In some embodiments, the presently disclosed kits include one or more containers, including, but not limited to a vial, tube, ampule, bottle, and the like, containing the 12-LO inhibitor. The presently disclosed kits or pharmaceutical systems also can include associated instructions for using the 12-LO inhibitor containing compositions for treating of COVID-19.

### BRIEF DESCRIPTION OF THE DRAWINGS

Various embodiments of the present disclosure are described herein with reference to the drawings.

FIG. 1A-D. FIG. 1A shows lung inflammatory pathology in COVID-19 lung (H&E staining) indicating the areas where 12-LO staining has been seen. FIG. 1B shows a lung with acute lung injury/diffuse alveolar damage. FIG. 1C shows thickened alveolar walls lined by hyaline membranes and intravascular fibrin aggregate (circle). FIG. 1D shows lung without acute lung injury.

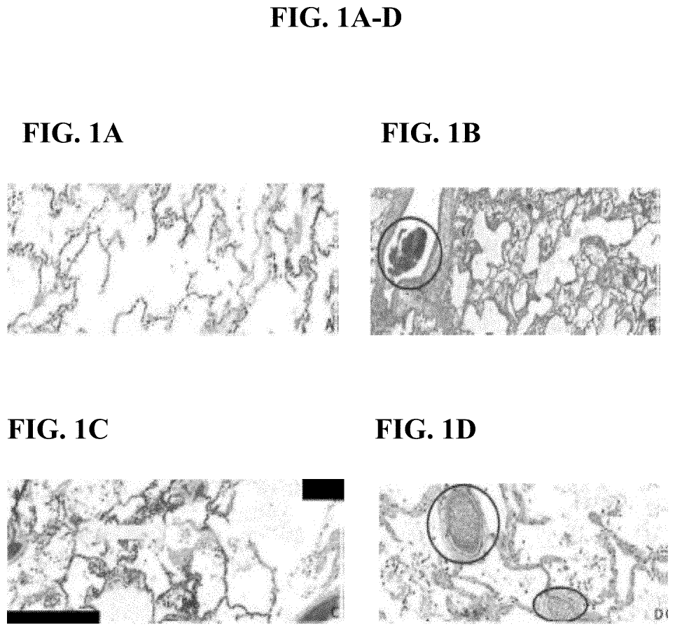

FIG. 2 shows actions of 12-lipoxygenase (12-LO) and its major product, 12-HETE, in the in the B-cell, and the extracellular pathways that regulate 12-LO.

FIG. 3A-D show 12-LO protein expression in three formalin fixed paraffin embedded samples of lung tissue from decedents who had RT-PCR confirmed COVID-19: negative control lung sample (FIG. 3A) with near absence of 12-LO staining; from COVID-19 lungs with 12-LO staining in pulmonary embolus (FIG. 3B); alveolar lung parenchyma (FIG. 3C); and vessel wall (FIG. 3D). DAPI staining is used to indicate cell nuclei.

FIG. 4 shows evidence of selective ALOX12 protein expression in islet from COVID-19 positive decedent.

FIG. 5. shows evidence of selective ALOX12 protein expression in lung tissue after SARS-CoV-2 infection in the K-18hACE2 transgenic mouse.

### DETAILED DESCRIPTION

The presently disclosed subject matter provides a method for preventing, or alleviating, the symptoms associated with COVID-19 through administration of a 12-LO inhibitor in a therapeutically effective amount to a subject infected, or suspected of being infected, with SARS-CoV-2. While not being bound to any theory, it is believed that COVID-19 mediated expression of 12-LO, an inflammatory enzyme, within the lungs and blood vessels leads to cytokine activation. This is supported by the observed increased expression of 12-LO in the lungs and blood vessels of COVID-19 patients as compared to control. Accordingly, administration of a 12-LO inhibitor can be used to reduce the symptoms and severity associated with SARS-CoV-2 infection. The 12-LO inhibitor serves in this capacity by inhibiting 12-LO activity.

As used herein, the term “coronavirus” is meant to include all microorganisms classified and identified as coronavirus. There are hundreds of coronaviruses, most of which circulate among such animals as pigs, camels, bats, and cats. Coronaviruses are a large family of viruses that usually cause mild to moderate upper-respiratory tract illnesses, such as the common cold. However, coronaviruses have emerged from animal reservoirs over the past two decades to cause serious and widespread illness and death. Such coronaviruses include, for example, SARS coronavirus (SARS-CoV) causing severe acute respiratory syndrome (SARS), MERS coronavirus (MERS-CoV) causing Middle East respiratory syndrome (MERS) and SARS-CoV-2 causing coronavirus disease 2019 (COVID-19).

As used herein, in general, a “therapeutically effective amount” of a 12-LO inhibitor refers to the amount of the agent necessary to elicit the desired biological response. In a specific embodiment, an effective amount is an amount sufficient for inhibition, prevention or treatment of COVID-19 and associated symptoms.

The effective amount of an agent may vary depending on such factors as the desired biological endpoint, the composition of the pharmaceutical composition, the target tissue or cell, the health of the subject to be treated and the like. In some embodiments, the term “therapeutic effective amount” refers to an amount sufficient to reduce or ameliorate the severity, duration, progression, or onset of symptoms associated with COVID-19.

A “subject” can include a human subject for medical purposes, such as for the treatment of COVID-19 or the prophylactic treatment for preventing the onset of a COVID-19, or an animal subject for medical, veterinary purposes, or developmental purposes. Further, a “subject” can include a patient afflicted with or suspected of being afflicted with COVID-19. Thus, the terms “subject” and “patient” are used interchangeably herein. In one embodiment, the presently disclosed subject matter relates to a method of treating or preventing COVID-19 in a subject in need thereof, the method including oral administration to the subject of a therapeutically effective amount of a 12-LO inhibitor.

In a non-limiting embodiment, the “subject” may be one having one or comorbidities or underlying conditions that may affect the severity of COVID-19. Such comorbidities or underlying conditions include, but are not limited to cancer, chronic kidney disease, Pulmonary disease, heart conditions, immunocompromised states, severe obesity, pregnancy, type 1 or 2 diabetes mellitus, cerebrovascular disease, liver disease and neurologic conditions.

As used herein, “COVID-19” means that the subject has symptoms associated with SARS-CoV-2 infection. Such COVID-19 symptoms include, for example, fever, chills, cough, shortness of breath or difficulty breathing, fatigue, muscle or body aches, headache, new loss of taste or smell, sore throat, congestion or runny nose, nausea or vomiting and diarrhea.

As used herein, the term “inhibit”, “inhibition” or “inhibits” means to decrease, suppress, attenuate, diminish, arrest, or stabilize the development or progression of COVID-19 by at least 10%, 20%, 40%, 40%, 50%, 60%, 70%, 80%, 90%, 95%, 98%, 99%, or even 100% compared to an untreated control subject. By the term “decrease” is meant to inhibit, suppress, attenuate, diminish, arrest, or stabilize a symptom of COVID-19. It should be appreciated that treating a disease, disorder or condition does not require that the disease, disorder, condition, or symptoms associated therewith be completely eliminated.

As used herein, the terms “treat,” treating,” “treatment,” and the like, are meant to decrease, suppress, attenuate, diminish, arrest, the underlying cause of COVID-19 or to stabilize the development or progression of COVID-19 and/or symptoms associated therewith. The terms “treat,” “treating,” “treatment,” and the like, as used herein can refer to curative therapy, prophylactic therapy, and preventative therapy. Accordingly, as used herein, “treating” means either slowing, stopping or reversing the progression of COVID-19, including reversing the progression to the point of eliminating the symptoms of COVID-19.

As used herein, the terms “prevent,” “preventing,” “prevention,” “prophylactic treatment” and the like refer to reducing the probability of developing symptoms of COVID-19 in a subject, who does not have, but is at risk of or susceptible to developing COVID-19 due to exposure to a SARS-CoV-2 infected subject. Thus, in some embodiments, a 12-LO inhibitor can be administered prophylactically to prevent the onset of COVID-19 or to prevent the recurrence of COVID-19 in a subject.

The treatment, administration, or therapy can be continuous or intermittent. Continuous treatment, administration, or therapy refers to treatment on at least a daily basis without interruption in treatment by one or more days. Intermittent treatment or administration, or treatment or administration in an intermittent fashion, refers to treatment that is not continuous, but rather cyclic in nature. Treatment according to the presently disclosed methods can result in complete relief or cure from COVID-19 or partial amelioration of one or more symptoms of a COVID-19 and can be temporary or permanent.

In certain embodiments, the presently disclosed subject matter also includes combination therapies. Additional therapeutic agents, which are normally administered to treat or prevent a COVID-19 infection, may be administered in combination with a 12-LO inhibitor as disclosed herein. For example, the 12-LO inhibitor may optionally be administered in conjunction with other compounds (e.g., therapeutic agents) or treatments useful in treating COVID-19. These additional agents may be administered separately, as part of a multiple dosage regimen, from the composition comprising a 12-LO inhibitor as disclosed herein. Alternatively, these agents may be part of a single dosage form, mixed together with a 12-LO inhibitor, in a single composition.

By “in combination with” is meant the administration of a 12-LO inhibitor, with one or more therapeutic agents either simultaneously, sequentially, or a combination thereof. Therefore, a subject can be administered a combination of a 12-LO inhibitor and one or more therapeutic agents at the same time (i.e., simultaneously) or at different times (i.e., sequentially, in either order, on the same day or on different days), so long as the effect of the combination of both agents is achieved in the subject. Where the 12-LO inhibitor and one or more therapeutic agents are administered simultaneously, they can be administered to the subject as separate pharmaceutical compositions, each containing either a 12-LO inhibitor or one or more therapeutic agents or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

Pharmaceutical compositions and formulations for use in treatment of COVID-19 include pharmaceutical compositions of a 12-LO inhibitor, alone or in combination with one or more additional therapeutic agents, in a mixture with a physiologically compatible carrier, which can be administered to a subject, for example, a human subject, for therapeutic or prophylactic treatment. Such 12-LO inhibitors include, for example, cinnamyl-3,4-dihydroxy-α-cyanocinnamate (CDC), 5,6,7-trikydroxyflavone (baicalein), N-benzyl-N-hydroxy-5-phenylpentamide (BHPP) and 8-hydroquinoline compounds, caffeic acid derivatives, 3-methoxytropolone and ML355 (Veralox Therapeutics) to name a few. (See also, Tsai et al, 2021, Biorg. Med Chem 46:116347).

In a specific embodiment, pharmaceutical compositions are provided comprising nucleic acid molecules designed to target 12-LO mRNA and inhibit, silence or attenuated the expression of that RNA for use in methods for prevention or treatment of COVID-19. The terms “inhibit,” “silencing,” and “attenuating” can refer to a measurable reduction in expression of a target mRNA (or the corresponding polypeptide or protein) as compared with the expression of the target mRNA (or the corresponding polypeptide or protein) in the absence of an interfering RNA molecule. The reduction in expression of the target mRNA (or the corresponding polypeptide or protein) is commonly referred to as “knock-down” and is reported relative to levels present following administration or expression of a non-targeting control RNA.

The term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways, wherein the compounds interfere with gene expression.

The term “shRNA” refers to any small hairpin RNA or short hairpin RNA. Although it is not necessary to understand the mechanism of action, it is believed that any sequence of RNA that makes a tight hairpin turn can be used to silence gene expression via RNA interference. Typically, shRNA uses a vector introduced into a cell genome and is constitutively expressed by a compatible promoter. The shRNA hairpin structure may also be cleaved into siRNA, which may then become bound to the RNA-induced silencing complex (RISC). This complex binds to and cleaves mRNAs which match the siRNA that is bound to it.

The term “microRNA” or “miRNA”, refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression. Methods for design and expression of such nucleic acids, e.g., antisense, miRNA, siRNA and shRNA, are well known to those of skill in the art. The ALOX-12 gene encodes for arachidonic acid 12-lipoxygenase. Accordingly, such inhibitory nucleic acids may be designed based on the publicly available sequence of the ALOX-12 gene. In an embodiment of the invention, the ALOX-12 gene is a human gene. (See, for example, ALOX12 arachidonate 12-lipoxygenase, 12S type (Homo sapiens) (See, Pub Med Gene ID: 239 and references cited therein).

In an embodiment, antibody molecules that bind to 12-LO may also be used inhibit the activity of 12-LO in COVID-19 subjects. “Antibody molecule” as used herein is intended to include intact antibodies, such as polyclonal antibodies or monoclonal antibodies (mAbs), as well as proteolytic fragments thereof such as the Fab or F(ab′)₂ fragments, chimeric antibodies, nanobodies, recombinant and engineered antibodies, single-chain antibodies and fragments thereof, as well as other molecules having at least one 12-LO antigen-binding site.

Also provided is a nanoparticle comprising a 12-LO inhibitor for use in treatment of COVID-19. Such nanoparticles can be natural or synthetic and may be incorporated into a vaccine composition. They can be created from biological molecules or from non-biological molecules. In some cases, the protein complex is crosslinked to a polymer or lipid on nanoparticle surface. In embodiments, the protein complex is adsorbed onto the nanoparticle surface. In some embodiments, the protein complex is adsorbed onto the nanoparticle surface and then crosslinked to the nanoparticle surface. In some embodiments, the protein complex is encapsulated into the nanoparticle.

In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof. In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000] (DPPE-PEG2000), 1,2-distearoyl-sn-glycero-3-phosphoethanolamine-N-[methoxy(polyethylene glycol)-1000] (DSPE-PEG2000), and cholesterol.

In some embodiments, the 12-LO inhibitor is coated on the nanoparticle using a crosslinking agent. In some embodiments, the 12-LO inhibitor is adsorbed onto the nanoparticle surface. In some embodiments, the 12-LO inhibitor is adsorbed onto the nanoparticle surface followed by covalent crosslinking of the 12-LO inhibitor to the nanoparticle surface using a crosslinking agent.

Crosslinking agents suitable for crosslinking the 12-LO inhibitor to produce the nanoparticle, or to coat SC-membrane protein on the nanoparticle are known in the art, and include those selected from the group consisting of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, and Sulfo-EGS.

Pharmaceutical compositions and formulations for use in treatment of COVID-19 include pharmaceutical compositions comprising an effective amount of a 12-LO inhibitor and a physiologically compatible carrier, which can be administered to a subject, for example, a human subject, for therapeutic or prophylactic treatment of COVID-19. As used herein, “physiologically compatible carrier” refers to a physiologically acceptable diluent including, but not limited to water, phosphate buffered saline, or saline, and, in some embodiments, can include an adjuvant. Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrans; chelating agents such as EDTA; sugar alcohols such as mannitol or sorbitol; salt-forming counter-ions such as sodium; and/or nonionic surfactants such as Tween, Pluronics, or PEG. Adjuvants suitable for use with the presently disclosed compositions include adjuvants known in the art including, but not limited to, incomplete Freund's adjuvant, aluminum phosphate, aluminum hydroxide, and alum.

The presently disclosed pharmaceutical compositions can be administered using a variety of methods known in the art. More particularly, as described herein, the 12-LO inhibitor can be administered to a subject for treatment of COVID-19 by any suitable route of administration, including orally, nasally, transmucosally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments, including buccally and sublingually, transdermally, through an inhalation spray, or other modes of delivery known in the art.

In one embodiment, the present disclosure provides a method for treating the respiratory system of a subject comprising forming an aerosol of a dispersion of particles, wherein the particles comprise a 12-LO inhibitor and an additive that enhances absorption of the drug into tissue of the respiratory system and administering the aerosol to the respiratory system of the subject. Pulmonary drug delivery of the 12-LO inhibitor is accomplished by inhalation of an aerosol through the mouth and throat. In an embodiment, the pharmaceutical compositions may be formulated for pulmonary delivery. Optimized formulations for such delivery may include addition of permeability enhancers (mucoadhesives, nanoparticles, and the like) as well as combined use with a pulmonary drug delivery device (for example, one that provides controlled particle dispersion with particles aerosolized to target the upper nasal cavity).

Pharmacologically active amounts of the drug substance, i.e., 12-LO inhibitors, are thereby delivered to the circulation or directly to the site of action, i.e., the lung. The present disclosure relates to a dosage form (for example a nasal spray, a nasal gel, a nasal ointment, inhalation solutions, inhalation suspensions, inhalation sprays, dry powder or an aerosol) which is specifically designed or adapted for administration of a drug substance to the nasal structures. Hence in one aspect the disclosure relates to the 12-LO inhibitors disclosed herein for use in a therapeutic method, wherein the drug substance is administered intranasally. In an embodiment, administration is to the upper nasal cavity. The pharmaceutical compositions may be delivered using an inhalator.

A pharmaceutical composition may be prepared, packaged, or sold in a formulation suitable for pulmonary administration via the buccal cavity. Such a formulation may include dry particles that contain the active ingredient and have a diameter in the range from about 0.5 to about 7 nanometers, and in certain embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device including the active ingredient dissolved or suspended in a low-boiling propellant in a sealed container.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further contain additional ingredients such as a liquid non-ionic or solid anionic surfactant or a solid diluent (in certain embodiments having a particle size of the same order as particles comprising the active ingredient).

Pharmaceutical compositions formulated for pulmonary delivery may also provide the 12-LO inhibitor in the form of droplets of a solution or suspension. Such formulations may be prepared, packaged, or sold as aqueous or dilute alcoholic solutions or suspensions, optionally sterile, including the active ingredient, and may conveniently be administered using any nebulization or atomization device. Such formulations may further include one or more additional ingredients including, but not limited to, a flavoring agent such as saccharin sodium, a volatile oil, a buffering agent, a surface active agent, or a preservative such as methylhydroxybenzoate. The droplets provided by this route of administration in certain embodiments have an average diameter in the range from about 0.1 to about 200 nanometers.

In one aspect, the 12-LO inhibitor is administered orally. When a therapeutically effective amount of the composition(s) is administered orally, it may be in the form of a solid or liquid preparation such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition(s) may contain a solid carrier, such as a gelatin or an adjuvant. The tablet, capsule, and powder may contain from about 0.05 to about 95% of the 12-LO inhibitor compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition(s) may further contain physiological saline solution, dextrose or other saccharide solution, or glycols such as ethylene glycol, propylene glycol, or polyethylene glycol.

In another embodiment, the composition(s) of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the composition(s) in an aqueous or non-aqueous pharmaceutically acceptable solvent which may also contain suspending agents, sweetening agents, flavoring agents, and preservative agents as are known in the art.

In some embodiments, the presently disclosed pharmaceutical compositions can be administered by rechargeable or biodegradable devices. For example, a variety of slow-release polymeric devices have been developed and tested in vivo for the controlled delivery of drugs. Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A). Sustained release compositions also include liposomally entrapped a 12-LO inhibitor, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy. Such materials can include an implant, for example, for sustained release of the 12-LO inhibitor.

The presently disclosed subject matter also includes the use of a 12-LO inhibitor, in the manufacture of a medicament for treatment of COVID-19. Regardless of the route of administration selected, the 12-LO inhibitor pharmaceutical compositions are formulated into pharmaceutically acceptable dosage forms such as described herein or by other conventional methods known to those of skill in the art.

Actual dosage levels of the 12-LO inhibitor can be varied to obtain an amount of the active ingredient that is effective to achieve the desired therapeutic response for a particular subject, composition, route of administration, and disease, disorder, or condition without being toxic. The selected dosage level will depend on a variety of factors including the route of administration, the time of administration, the rate of excretion, the duration of the treatment, other drugs used in combination with the 12-LO inhibitor, the age, sex, weight, condition, general health and prior medical history of the patient being treated, and like factors well known in the medical arts.

A physician having ordinary skill in the art can readily determine and prescribe the effective amount of a 12-LO inhibitor-containing pharmaceutical composition required. For example, the physician could start doses of the 12-LO inhibitor lower than that required to achieve the desired therapeutic effect and gradually increase the dosage until the desired effect is achieved. Accordingly, the dosage range for administration will be adjusted by the physician, as necessary. It will be appreciated that an amount of a 12-LO inhibitor required for achieving the desired biological response, e.g., treatment or prevention of COVID-19, may be different from the amount of a 12-LO inhibitor effective for another purpose.

In general, a suitable daily dose of a 12-LO inhibitor will be that amount of the drug that is the lowest dose effective to produce a therapeutic effect. Such an effective dose will generally depend upon the factors described above. Effective dosages may be determined based generally on the weight of the subject to be treated. If desired, the effective daily dose of the 12-LO inhibitor can be administered as one, two, three, four, five, six or more sub-doses administered separately at appropriate intervals throughout the day, optionally, in unit dosage forms.

In a non-limiting embodiment, a typical dosage may be in the range from about 0.0001 to 1000 mg/kg. The daily dose of the compound or salt thereof contained in the composition of the present invention may be about 0.0001 to 500 mg/kg of body weight, 0.001 to 50 mg/kg of body weight or 0.01 to 5 mg/kg. In other non-limiting examples, a dose may also comprise from about 1 μg/kg body weight, about 5 μg/kg body weight, about 10 μg/kg body weight, about 25 μg/kg body weight, about 50 μg/kg body weight, about 100 μg/kg body weight, about 250 μg/kg body weight, about 350 μg/kg body weight, about 500 μg/kg body weight, about 750 μg/kg body weight, about 1 mg/kg body weight, about 5 mg/kg body weight, about 10 mg/kg body weight, about 25 mg/kg body weight, about 50 mg/kg body weight, about 100 mg/kg body weight, about 250 mg/kg body weight, about 350 mg/kg body weight, about 500 mg/kg body weight, about 750 mg/kg body weight to about 1000 mg/kg body weight or more per administration, and any range derivable therein.

In an embodiment, pharmaceutical compositions containing a 12-LO inhibitor are provided for use in a method of treating or ameliorating the symptoms in a subject infected with COVID-19.

The presently disclosed compositions of a 12-LO inhibitor can be assembled into kits or pharmaceutical systems for use in treating or preventing COVID-19. In some embodiments, the presently disclosed kits or pharmaceutical systems include a 12-LO inhibitor in unit dosage form. In further embodiments, the 12-LO inhibitor can be present together with a pharmaceutically acceptable solvent, carrier, excipient, or the like, as described herein.

In some embodiments, the presently disclosed kits include one or more containers, including, but not limited to a vial, tube, ampule, bottle, and the like, for containing the 12-LO inhibitor. The one or more containers also can be carried within a suitable carrier, such as a box, carton, tube, or the like. Such containers can be made of plastic, glass, laminated paper, metal foil, or other materials suitable for holding medicaments.

The presently disclosed kits or pharmaceutical systems also can include associated instructions for using the 12-LO inhibitor containing compositions for treating COVID. In some embodiments, the instructions include one or more of the following: a description of pharmaceutical composition containing a 12-LO inhibitor; a dosage schedule and administration for treating or preventing COVID-19; precautions; warnings; indications; counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

### EXAMPLE

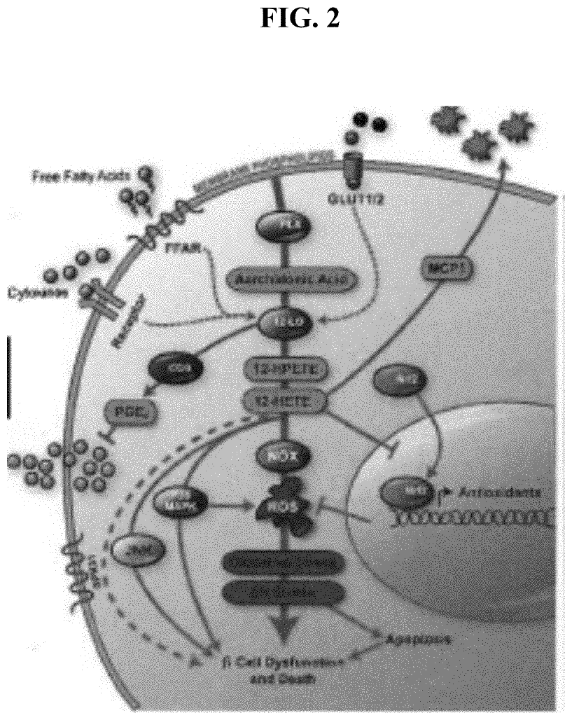

The lipoxygenases (LOs) form a family of enzymes that catalyze the oxygenation of cellular poly-unsaturated fatty acids to form lipid inflammatory mediators. For example, 12-lipoxygenase (12-LO) converts arachidonic acid (C20-4) to 12-hydroperoxyeicosatetraenoic acid (12-HETE) by glutathione peroxidase. In the islet, increased expression, or activity of 12-LO catalyzes the production of 12-HETE, which accelerates inflammation (via MAPK and NFkB pathways) and promotes oxidative stress, in part by preventing the action of the Nrf2 transcription factor (see, FIG. 2).

Studies have demonstrated that 12-LO plays an intrinsic role in islet inflammation and dysfunction. 12-LO has been identified in both the rodent and human endocrine pancreas, and in both are up-regulated further in the setting of pre-diabetes and frank T2D. It has been demonstrated that pancreas-specific 12-LO knockout mice exhibit protection from glucose intolerance during HFD feeding, suggesting that 13-cell-intrinsic 12-LO may be pivotal to glucose intolerance in obesity and T2D. Elimination of 12-LO in the mouse also protects against Type 1 diabetes by preventing immune-mediated 13 cell loss. In addition, a selective 12-LO inhibitor, ML-355, has been shown to protect human islets from inflammatory injury in vitro,

It has been shown that 12-LO and inflammatory products, such as 12-S-HETE lead to monocyte trafficking and binding to human endothelial cells. Further, 12-LO is expressed in vascular tissue. Several reviews have highlighted the role of 12-LO and products in leading to cardiovascular and other complications of diabetes, including endothelial dysfunction. Recent studies clearly indicate a major effect of the 12-LO product, 12-S-HETE to induce platelet hyperactivity and thrombosis. In addition, 12-LO products in the mouse lung induce macrophage polarization and chemokine production. Deletion of the 12-LO form in the mouse prevented neutrophil recruitment and improved lung function and structure in an acute lung injury model.

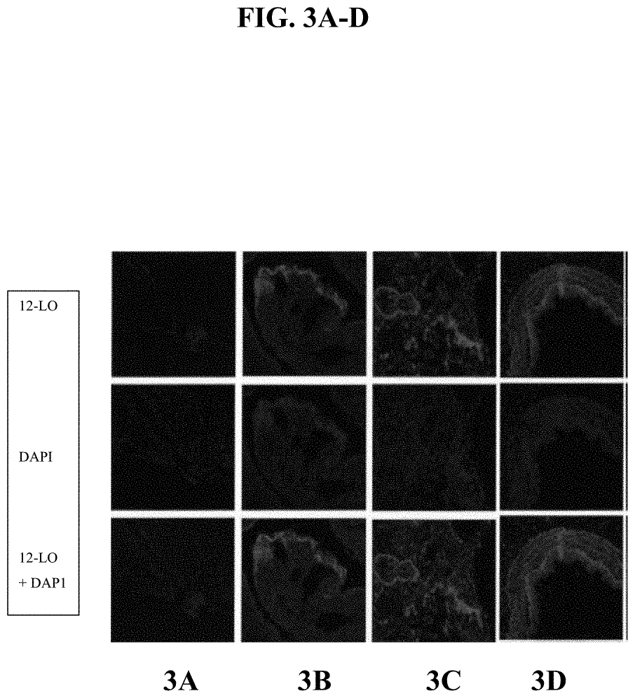

Analysis of 12-LO protein expression was done in three formalin fixed paraffin embedded samples of lung tissue from decedents who had RT-PCR confirmed COVID-19. A 12-LO antibody was used for immunofluorescence of 12-LO expression. These samples showed lung injury and in many cases with significant thrombosis. COVID-19 positive decedents had known diabetes or prediabetes. 12-LO expression was compared to levels seen in a control case from an individual who was COVID-19 negative and had a lung biopsy after a spontaneous pneumothorax. As demonstrated in FIG. 3, strong 12-LO staining was seen in the COVID-19 positive samples.

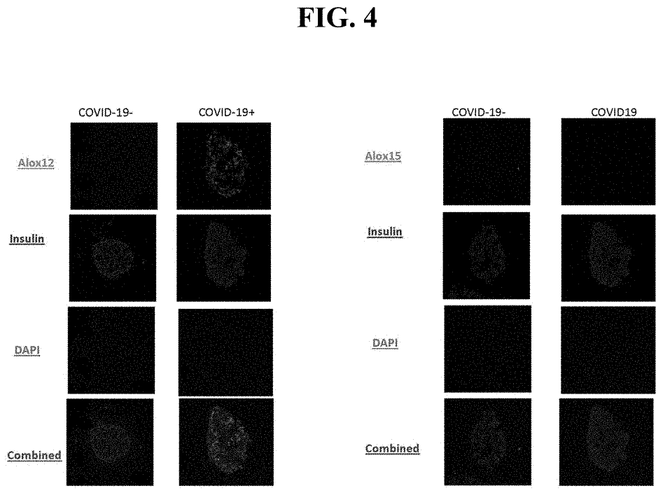

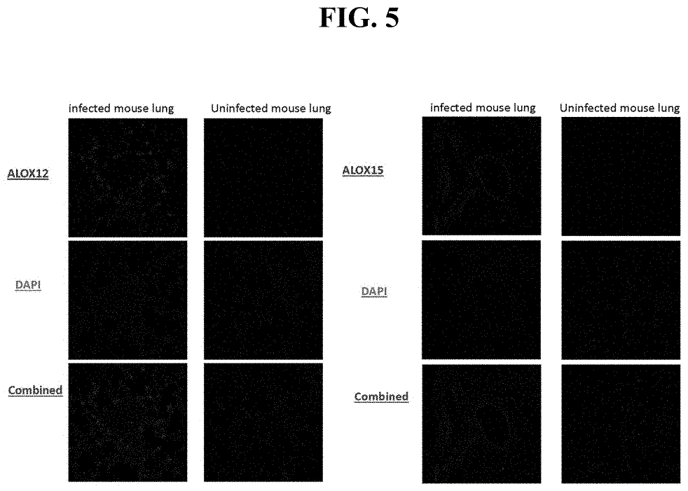

FIG. 4 depicts evidence of positive staining in the islet for ALOX12 (12-LO) protein in the decedent who had active COVID-19 infection compared to an autopsy section from a decedent who died from an unrelated respiratory illness. Interestingly, the staining in the COVID-19 positive sample is selective for ALOX12 and not ATOX15. Samples were also obtained for evaluating SARS-CoV-2 infection in the K18-hACE2 transgenic mouse model (K18-hACE2), which is an accepted mouse model that shows tissue changes characteristic of COVID-19. Lung tissue from acute high dose (lethal) virus infection was analyzed. The data shown in FIG. 5 indicates selective increase in ALOX12 protein but not ALOX15. This result is supports that in vivo SARS-CoV-2 infection in the K18-hACE2 mouse model increase 12-LO expression further supporting the importance of 12-LO inhibition to treat COVID-19.

Other embodiments of the present disclosure with be apparent to those skilled in the art from consideration of the present specification. Publications cited throughout this document are hereby incorporated by reference in their entirety. It is intended that the present specification and examples be considered as exemplary only with a true scope and spirit of the disclosure being indicated by the following claims and equivalents thereof. The examples above are presented to further illustrate selected embodiments of the present disclosure.

What is claimed:

1. A method of treating, or preventing, the symptoms associated with a coronavirus infection in a subject in need thereof, the method comprising administration to the subject of a therapeutically effective dose of a 12-LO inhibitor.

2. The method of claim 1, wherein the coronovirus infection is a SARS-COV-2 infection.

3. The method of claim 1, wherein the subject has COVID-19.

4. The method of claim 1, wherein the 12-LO inhibitor is a small molecule.

5. The method of claim 1, wherein the 12-LO inhibitor is ML355.

6. The method of claim 1, wherein the 12-LO inhibitor is a 12-LO antibody.

7. The method of claim 1, wherein the 12-LO inhibitory is an inhibitory nucleic acid that targets the ALOX-12 gene product.

8. The method of claim 1, wherein the 12-LO inhibitor is administered orally, intranasally, by inhalation and/or intravenously.

9. The method of claim 1, wherein the 12-LO inhibitor is administered intranasally.

10. The method of claim 1, wherein the 12-LO is administer for pulmonary delivery.

11. The method of claim 1, wherein the 12-LO inhibitor is formulated into an aerosol of dispersion particles.

12. The method of claim 1, wherein the 12-LO inhibitor is administered to the subject intermittently.

13. The method of claim 1, wherein the 12-LO inhibitor is administered to the subject continuously.

14. The method of claim 1, further comprising administration of a second COVID-19 therapeutic agent.

15. A kit comprising: (i) one or more containers for containing a 12-LO inhibitor-containing compositions; and (ii) instructions for using the 12-LO inhibitor containing compositions for treatment of COVID-19.

16. The kit of claim 15, wherein the instructions include one or more of the following: (i) a description of pharmaceutical composition containing a 12-LO inhibitor; (ii) a dosage schedule; or (iii) instructions for administration of the pharmaceutical composition containing a 12-LO inhibitor for treatment of COVID-19.

17. A pharmaceutical composition, comprising a 12-LO inhibitor formulated for treatment of COVID-19.

18. The composition of claim 17, wherein the formulation is for pulmonary delivery.
